(19) United States
(12) Patent Application Publication
(10) Pub. No.: US 2006/0183143 A1
(43) Pub. Date: Aug. 17, 2006

(54) Method and apparatus for classifying nucleic acid responses to infectious agents

(76) Inventors: Patrick Denis Lincoln, Woodside, CA (US); Steven Mark Eker, Menlo Park, CA (US)

Correspondence Address: PATTERSON & SHERIDAN, LLP;SRI INTERNATIONAL, 595 SHREWSBURY AVENUE, SUITE 100, SHREWSBURY, NJ, 07702, US

(21) Appl. No.: 11/335,982

(22) Filed: Jan. 20, 2006

Related U.S. Application Data
(60) Provisional application No. 60/645,708, filed on Jan. 20, 2005.

(51) Int. Cl. C12Q 1/68; G06F 19/00

(52) U.S. Cl. 435/6; 702/20

(57) ABSTRACT

In one embodiment, the present invention is a method and apparatus for classifying nucleic acid responses to infectious agents. In one embodiment, a method for selecting genes to be analyzed to determine exposure to a condition (from among a plurality of potential conditions) includes determining, for each gene in a set of test data that includes genes and corresponding expression patterns for exposure to given conditions, a distance between each pair of conditions. A subset of genes from within the set of test data is then identified for which the distance between each pair of conditions is maximized. In this way, the number of genes whose expression patterns must be analyzed in order to reliably diagnose a condition is minimized.

### CROSS REFERENCE TO RELATED APPLICATIONS

This application claims the benefit of U.S. Provisional Patent Application Ser. No. 60/645,708, filed Jan. 20, 2005, which is herein incorporated by reference in its entirety.

### REFERNCE TO GOVERNMENT FUNDING

This invention was made with Government support under contract number F30602-01-C-0153 awarded by the Air Force Research Laboratory. The Government has certain rights in this invention.

### FIELD OF THE INVENTION

The present invention relates generally to the health services field and relates more particularly to the detection of exposure to biological agents.

### BACKGROUND OF THE INVENTION

It has been proposed that an examination of messenger ribonucleic acid (mRNA) levels in an individual's blood or tissue may facilitate a diagnosis of an individual's health status, even before physical manifestations of the individual's health status are observable. Specifically, the patterns of mRNA expression in immune system cells (e.g., white blood cells) record and express information that may enable the identification of an infectious agent (e.g., a biowarfare agent, a virus, an allergen, etc.) to which the individual has been exposed, as well as the time since the exposure occurred.

The human gene set comprises tens of thousands of genes, which unfortunately makes monitoring the expression levels of all genes in an immune system cell impractical due to cost and time considerations. Effective and less costly analysis could be performed by monitoring only a fraction of the total gene set (e.g., a few hundred genes); however, the problem then becomes selecting the subset of genes that will produce the most meaningful results.

Thus, there is a need in the art for a method and apparatus for classifying nucleic acid responses to infectious agents.

### SUMMARY OF THE INVENTION

In one embodiment, the present invention is a method and apparatus for classifying nucleic acid responses to infectious agents. In one embodiment, a method for selecting genes to be analyzed to determine exposure to a condition (from among a plurality of potential conditions) includes determining, for each gene in a set of test data that includes genes and corresponding expression patterns for exposure to given conditions, a distance between each pair of conditions. A subset of genes from within the set of test data is then identified for which the distance between each pair of conditions is maximized. In this way, the number of genes whose expression patterns must be analyzed in order to reliably diagnose a condition is minimized.

### BRIEF DESCRIPTION OF THE DRAWINGS

The teaching of the present invention can be readily understood by considering the following detailed description in conjunction with the accompanying drawings, in which:

FIG. 1 is a flow diagram illustrating one embodiment of a method for selecting a subset of genes from within a set of test data for gene expression analysis;

FIG. 2 is a flow diagram illustrating one embodiment of a method for classifying an unknown sample (e.g., a blood sample from an individual) in accordance with gene expression analysis for a subset of the genes contained therein;

FIG. 3 is a flow diagram illustrating another embodiment of a method for calculating the distance between a first condition and a second condition relative to a given gene; and

FIG. 4 is a high level block diagram of the gene selection method that is implemented using a general purpose computing device.

To facilitate understanding, identical reference numerals have been used, where possible, to designate identical elements that are common to the figures.

### DETAILED DESCRIPTION

In one embodiment, the present invention relates to the classification of nucleic acid responses to infectious agents. Embodiments of the invention optimize the selection of a subset of genes (e.g., from a set of all genes within a human immune system cell) for gene expression analysis, where the ultimate goal of the analysis may be to identify a prevailing condition in a sample of an individual's blood. Probing only the genes in this reduced subset will allow diagnostic tests to be performed in a relatively inexpensive and timely manner, while maintaining the ability to reliably differentiate between different exposure conditions.

Within the context of the present invention, a “condition” is defined as at least an infectious agent (e.g., a biowarfare agent, a virus, an allergen, etc.) to which an individual has been exposed. In some embodiments, the condition additionally identifies the length of time since the individual was exposed to the infectious agent. Thus, for example, a condition may indicate exposure to influenza and may additionally indicate that the exposure took place approximately twenty-four hours ago.

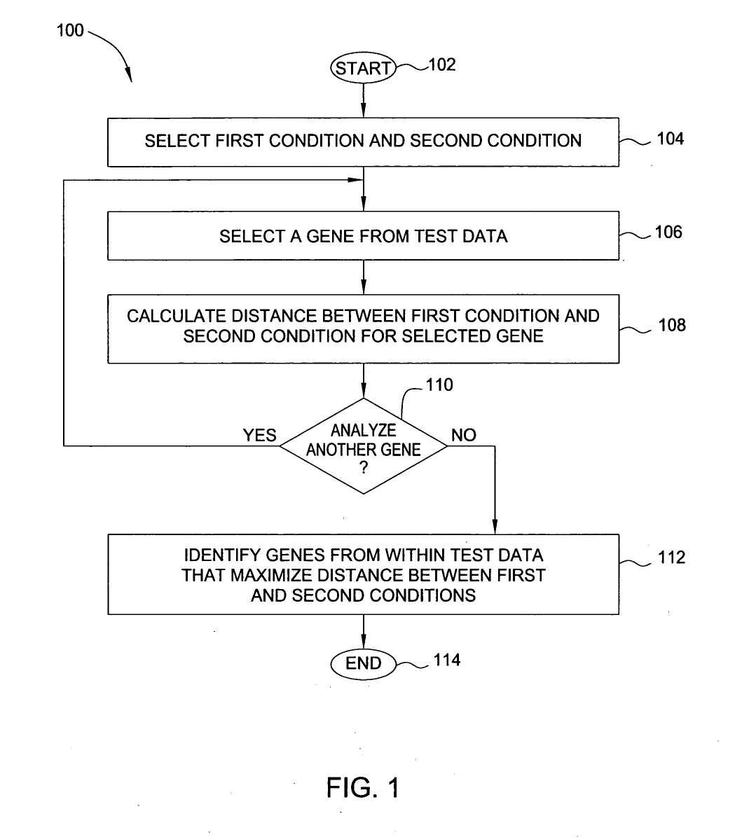

FIG. 1 is a flow diagram illustrating one embodiment of a method 100 for selecting a subset S of genes from within a set of test data for gene expression analysis. The test data may comprise, for example, gene expression patterns for given conditions for a set of individuals. The size of the subset, S, to be selected from the test data, may be dictated a priori (e.g., by the capabilities of a gene regulation device to be fielded), or may be selected in accordance with a stochastic approach. For example, noisy data with a level of randomness could be generated. The size, n, of the subset S could then be plotted versus the number of correct diagnoses, so that the optimal size, n, can be identified. The shapes of graphs plotted in this manner tend to improve the chances of selecting an optimal subset size, n, as n tends to increase up to some value, k, where the diagnosis accuracy levels off, before falling (e.g., due to the inclusion of genes with poor discriminatory power). Thus, values of n that are equal to or slightly greater than k may be sensible choices. This approach can be performed for different levels of randomness.

The method 100 is initialized at step 102 and proceeds to step 104, where the method 100 selects a pair of conditions (e.g., a first condition and a second condition) from among a set of conditions to be detected. For example, the first condition might be anthrax exposure within twenty-four hours and the second condition might be influenza exposure within twenty-four hours.

In step 106, the method 100 selects a gene from within the test data for analysis. The method 100 then proceeds to step 108 and calculates the distance between the first condition and the second condition for the selected gene. In one embodiment, the distance is a set theoretic distance function, where the distance between the first condition and the second condition is calculated by first determining first and second regulation types for the selected gene with regard to the first condition and the second condition, respectively. That is, the method 100 determines, for each of the first condition and the second condition, whether exposure thereto results in the selected gene being upregulated, downregulated and/or unchanged (i.e., versus a pre-exposure condition of the gene). The method 100 then compares the first regulation type and the second regulation type for the selected gene, and assigns a score to the gene based on this comparison.

In one embodiment, regulation types for the selected gene are regarded as subsets of {up, down, same}, and distance between conditions is scored on scale of zero to three, where zero represents the smallest possible distance and three represents the largest possible distance. In one embodiment, it is assumed that each regulation condition is equally likely for a given gene. Thus, if the first regulation type and the second regulation type are identical, the method 100 assigns a lowest distance (e.g., of zero) between the first condition and the second condition for the selected gene (i.e., the post-exposure regulation type of the selected gene does not allow unambiguous differentiation between the first and second condition); if the first regulation type and the second regulation type for the selected gene are disjoint (no elements in common), the method 100 assigns a highest distance (e.g., of three) between the first condition and the second condition for the selected gene (i.e., the post-exposure regulation type of the selected gene allows unambiguous differentiation between the first and second condition). Additionally, if one of the first regulation type and the second regulation type is a subset of the other, the method 100 assigns a second-lowest distance (e.g., of one) between the first condition and the second condition for the selected gene; if neither of the first regulation type and the second regulation type is a subset of the other, the method 100 assigns a second-highest distance (e.g., of two) between the first condition and the second condition for the selected gene.

In an alternative embodiment, the distance is a bit-wise distance function, where regulation types for genes are regarded as three-bit vectors with the bit positions corresponding to “upregulated”, “downregulated” and “same” (unchanged). The distance function returns the Hamming distance, H (e.g., the number of positions at which corresponding elements of the first condition and second condition differ, or the number of substitutions required to change the first condition into the second condition), between the bit positions, where 0≦H≦3. In one embodiment, two regulation types are considered to differ for the purpose of calculating the Hamming distance if, and only if, they have no overlap. The regulation of the selected gene is considered to be consistent with a bit vector if the corresponding bit (i.e., upregulated, downregulated or same) is one. The Hamming distance value (i.e., zero to three) represents the number of regulation values (drawn from upregulated, downregulated and same) for which the selected gene is consistent with exactly one of the first condition and the second condition. The intuition is that if the selected gene has a value that is consistent with exactly one of the first condition and the second condition, the gene can be used to distinguish between the two conditions.

In step 110, the method 100 determines whether to analyze another gene from the test data, i.e., to determine how well the gene will allow differentiation between the first condition and the second condition. In one embodiment, each gene in the test data is analyzed; thus, if any genes in the test data have not yet been analyzed, the method 100 proceeds to analyze a next gene in the test data. If the method 100 concludes in step 110 that another gene in the test data should be tested, the method 100 returns to step 106 and proceeds as described above in order to calculate the distance between the first condition and the second condition for the newly selected gene.

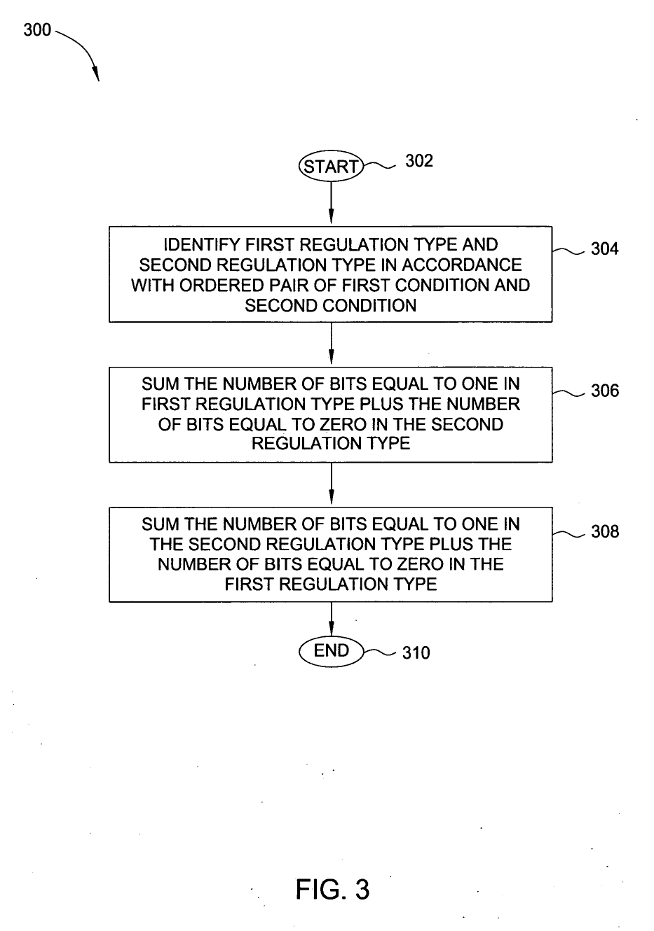

Alternatively, if the method 100 concludes in step 110 that no further genes in the test data need be analyzed, the method 100 proceeds to step 112 and identifies the subset, S, of analyzed gene(s) from within the test data that maximize the distance between the first condition and the second condition before terminating in step 114. This makes the first condition and the second condition as distinct as possible and maximizes the amount of error in the test data that can be tolerated. In one embodiment, for each unordered pair of conditions (where the first condition is different than the second condition), a value is computed that is sum of the distances between the first and second conditions (e.g., as calculated according to one of the methods described above) for all genes within a given subset, S. In one embodiment, the least of these sums for a subset, S, is considered to be representative of the subset's discriminatory power in general. In a more finely-grained embodiment, all of the sums are placed in a vector that is sorted in ascending order. Distance vectors for sets of genes are then compared lexicographically, where a bigger distance vector indicates better discriminatory capability. FIG. 3, discussed below, illustrate further embodiments of methods for calculating the distance between two conditions relative to a given gene. In one embodiment, a greedy hill-climbing algorithm is implemented to identify the optimal choice of gene(s).

In one embodiment, the method 100 identifies the subset, S, of genes in accordance with a “shrink” approach that starts with the complete set of genes in the test data and then removes genes, one at a time, such that the distance vector for the remaining set of genes is maximized. This process of removing genes from the set is repeated until the set is an empty set. Accordingly, the order in which genes were removed from the set indicates which genes are most useful for differentiating between conditions (i.e., the first-removed gene is the least useful, while the last-removed gene is the most useful). Thus, to select a subset, S, of the n most useful genes, the last n genes to be removed from the complete set are selected to form the subset, S.

In another embodiment, the subset, S, is chosen using a “grow” approach that starts with an empty set and then adds genes, one at a time, such that the distance vector in the enlarged set is maximized. This process of adding genes to the set is repeated until the set contains the complete set of genes. Accordingly, the order in which genes were added to the set indicates which genes are most useful for differentiating between conditions (i.e., the first-added gene is the most useful, while the last-added gene is the least useful). Thus, to select a subset, S, of the n most useful genes, the first n genes to be added to the set are selected to form the subset, S.

Thus, the method 100 identifies the genes that are most capable of differentiating between exposure to given conditions in an unambiguous manner. Therefore, when a subset of genes must be selected from a sample for diagnosis, the diagnosis can be optimized by performing gene expression analysis for only those genes that will provide the most reliable and unambiguous results. This reduces the cost and time associated with performing gene expression analysis for the sample, as genes whose expressions will provide little or no useful information will likely not be analyzed.

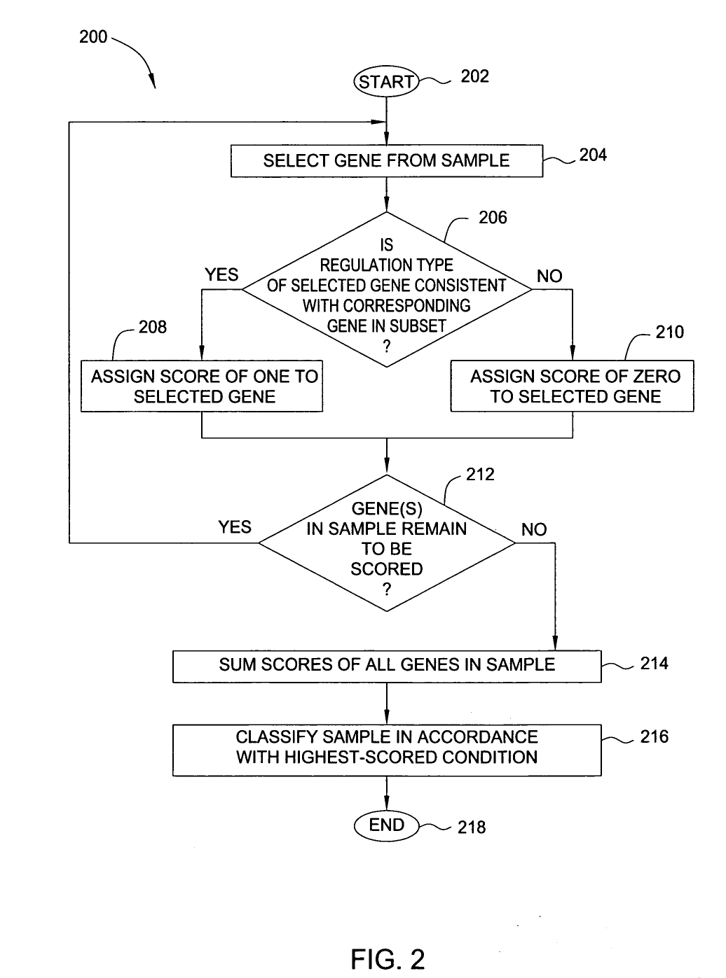

FIG. 2 is a flow diagram illustrating one embodiment of a method 200 for classifying an unknown sample (e.g., a blood sample from an individual) in accordance with gene expression analysis for a subset of the genes contained therein. The method 200 is initialized at step 202 and proceeds to step 204, where the method 200 selects a gene from the sample. The selected gene corresponds to a gene in a subset, S, of genes selected for gene expression analysis (e.g., as selected in accordance the method 100).

In step 206, the method 200 determines whether the regulation type of the selected gene (e.g., upregulated, downregulated or unchanged) is consistent with the regulation type of the corresponding gene in the subset, S. Remember that the regulation type of the corresponding gene in the subset, S, helps to differentiate between potential exposure conditions. If the method 200 concludes in step 206 that the regulation type of the selected gene is consistent with the regulation type of the corresponding gene in the subset, S, the method 200 proceeds to step 208 and assigns a maximum score to the selected gene. In one embodiment, the maximum score is one.

Alternatively, if the method 200 concludes in step 206 that the regulation type of the selected gene is not consistent with the regulation type of the corresponding gene in the subset, S, the method 200 proceeds to step 210 and assigns a minimum score to the selected gene. In one embodiment, the minimum score is zero.

Once the selected gene has been scored in accordance with step 208 or step 210, the method 200 proceeds to step 212 and determines whether there are any genes in the sample that remain to be scored. If the method 200 concludes in step 212 that there is at least one gene in the sample that remains to be scored, the method 200 returns to step 204 and proceeds as described above to score a next gene in the sample.

Alternatively, if the method 200 concludes in step 212 that there are no genes in the sample that remain to be scored, the method 200 proceeds to step 214 and sums the scores of all scored genes in the sample (which correspond to the genes in the subset, S).

In step 216, the method 200 classifies the sample in accordance with the highest-scored conditions. That is, the condition that corresponds to the highest cumulative score is selected as a condition to which the individual from which the sample came has likely been exposed. The method 200 then terminates in step 218.

FIG. 3 is a flow diagram illustrating another embodiment of a method 300 for calculating the distance between a first condition and a second condition relative to a given gene. The method 300 may be implemented, for example, in accordance with step 108 of the method 100 in order to facilitate the selection of a subset, S, of genes for gene expression analysis.

The method 300 leverages the observation that, for a given pair of conditions (e.g., a first condition and a second condition), the ability of a gene to correctly select the first condition over the second condition is not necessarily equivalent to the gene's ability to select the second condition over the first condition. For example, if the gene's regulation type for the first condition is USD (upregulated, same, downregulated), and the gene's regulation type for the second condition is US (upregulated, same), there is one case (downregulated) for which the gene can select the first condition over the second condition, but no cases where the gene can select the second condition over the first condition. Thus, if there are no genes in a subset that can ever select the second condition over the first condition, the second condition cannot be unambiguously recognized.

The method 300 is initialized at step 302 and proceeds to step 304, where the method 300 identifies, for the given gene, a first regulation type and a second regulation type. As described above with respect to the method 100, the first regulation type indicates the manner in which the gene is regulated in response to exposure to a first condition, whereas the second regulation type indicates the manner in which the gene is regulated in response to exposure to a second condition. However, in this case, the first condition and the second condition are an ordered pair, where the first condition is different from the second condition.

In step 306, the method sums the number of bits that are one in the first regulation type and the number of bits that are zero in the second regulation type. This gives the distance from the first regulation type to the second regulation type. The method 300 then proceeds to step 308 and sums the number of bits that are one in the second regulation type and the number of bits that are zero in the first regulation type. This gives the distance from the second regulation type to the first regulation type. This distance metric is not symmetric; i.e., the distance from the first regulation type to the second regulation type is not necessarily equal to the distance from the second regulation type to the first regulation type. Thus, the resultant distance vectors are exactly twice the length of the distance vectors produced in accordance with the method described in connection with FIG. 1 and now contain an entry for each ordered pair of conditions where the first condition is different from the second condition.

The method 300 terminates in step 310. The distance vectors produces in accordance with the method 300 can then be summed as discussed above.

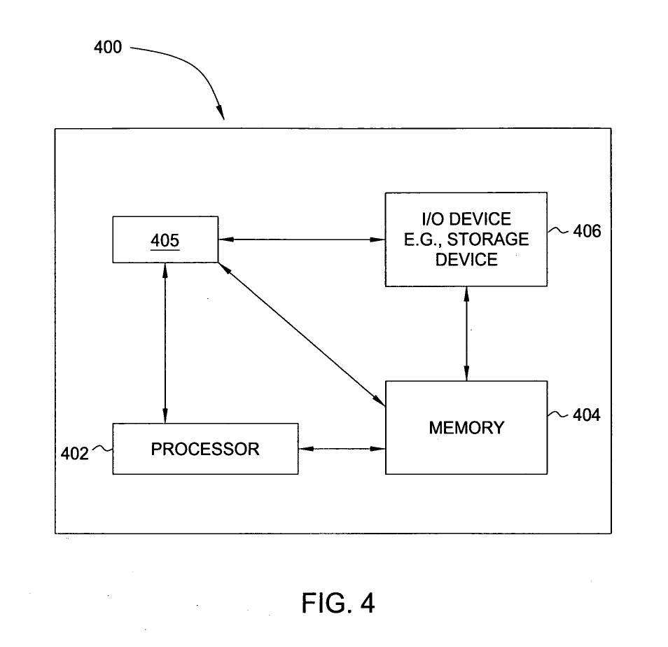

FIG. 4 is a high level block diagram of the gene selection method that is implemented using a general purpose computing device 400. In one embodiment, a general purpose computing device 400 comprises a processor 402, a memory 404, a gene selection module 405 and various input/output (I/O) devices 406 such as a display, a keyboard, a mouse, a modem, and the like. In one embodiment, at least one I/O device is a storage device (e.g., a disk drive, an optical disk drive, a floppy disk drive). It should be understood that the gene selection module 405 can be implemented as a physical device or subsystem that is coupled to a processor through a communication channel.

Alternatively, the gene selection module 405 can be represented by one or more software applications (or even a combination of software and hardware, e.g., using Application Specific Integrated Circuits (ASIC)), where the software is loaded from a storage medium (e.g., I/O devices 506) and operated by the processor 402 in the memory 404 of the general purpose computing device 400. Thus, in one embodiment, the gene selection module 405 for selecting subsets of genes for gene expression analysis described herein with reference to the preceding Figures can be stored on a computer readable medium or carrier (e.g., RAM, magnetic or optical drive or diskette, and the like).

Thus, the present invention represents a significant advancement in the field of health services. Embodiments of the invention optimize the selection of a subset of genes (e.g., from a set of all genes within a human immune system cell) for gene expression analysis, where the ultimate goal of the analysis may be to identify a prevailing condition in a sample of an individual's blood. Probing only the genes in this reduced subset will allow diagnostic tests to be performed in a relatively inexpensive and timely manner, while maintaining the ability to reliably differentiate between different exposure conditions.

While various embodiments have been described above, it should be understood that they have been presented by way of example only, and not limitation. Thus, the breadth and scope of a preferred embodiment should not be limited by any of the above-described exemplary embodiments, but should be defined only in accordance with the following claims and their equivalents.

What is claimed is:

1. A method for selecting genes to be analyzed to determine exposure to a condition from among a plurality of potential conditions, the method comprising:
determining, for each gene in a set of test data comprising a plurality of genes and corresponding expression patterns for exposure to said plurality of potential conditions, a distance between each pair of conditions in said plurality of potential conditions; and
identifying a subset of genes from within said set of test data for which said distance between each pair of conditions is maximized.

2. The method of claim 1, wherein said distance is a set theoretic distance.

3. The method of claim 2, wherein said set theoretic distance is calculated by:
determining, for a given gene, a first regulation type associated with exposure to a first condition from said plurality of potential conditions;
determining, for said given gene, a second regulation type associated with exposure to a second condition from said plurality of potential conditions; and
scoring said gene in accordance with a comparison of said first regulation type and said second regulation type.

4. The method of claim 3, wherein said scoring comprises:
assigning a lowest score if said first regulation type and said second regulation type are identical;
assigning a highest score if said first regulation type and said second regulation type are disjoint;
assigning a second-lowest score if one of said first regulation type and said second regulation type is a subset of the other; and
assigning a second-highest score if neither of the first regulation type and the second regulation type is a subset of the other.

5. The method of claim 1, wherein said distance is a bit-wise distance.

6. The method of claim 5, wherein a regulation type for each gene is a vector comprising:
a first bit position corresponding to upregulation;
a second bit position corresponding to downregulation; and
a third bit position corresponding to no change.

7. The method of claim 1, wherein said distance is a Hamming distance.

8. The method of claim 1 wherein said subset of genes is identified in accordance with a greedy hill-climbing algorithm.

9. The method of claim 8, wherein said greedy hill-climbing algorithm comprises:
starting with a group comprising every gene in said set of test data;
removing genes from said group one at a time until said group is empty, based on which gene in said group, at a given time, yields a largest distance vector for a remaining set of genes; and
selecting a number of last-removed genes to comprise said subset.

10. The method of claim 8, wherein said greedy hill-climbing algorithm comprises:
starting with an empty group;
adding genes to said group one at a time, based on which gene in said group, at a given time, yields a largest distance vector for said group, which now comprises at least one gene; and
selecting a number of first-added genes to comprise said subset.

11. The method of claim 1, wherein each of said pairs of conditions is an ordered pair comprising a first condition and a second condition.

12. The method of claim 11, wherein a distance between said first condition and said second condition comprises a sum of:
a number of bits comprising one for a first regulation type associated with said first condition; and
a number of bits comprising zero for a second regulation type associated with said second condition.

13. The method of claim 12, wherein a distance between said second condition and said first condition is not necessarily equal to said distance between said first condition and said second condition.

14. The method of claim 1, wherein a general ability of said subset of genes to distinguish between any two conditions in said plurality of potential conditions is calculated by:
for each pair of conditions in said plurality of potential conditions, computing a distance therebetween relative to each gene in said subset;
for each pair of conditions, summing said distances over said subset;
identifying a smallest sum of said distances; and
associating said ability with said smallest sum.

15. A computer readable medium containing an executable program for selecting genes to be analyzed to determine exposure to a condition from among a plurality of potential conditions, where the program performs the steps of:
determining, for each gene in a set of test data comprising a plurality of genes and corresponding expression patterns for exposure to said plurality of potential conditions, a distance between each pair of conditions in said plurality of potential conditions; and
identifying a subset of genes from within said set of test data for which said distance between each pair of conditions is maximized.

16. The computer readable medium of claim 15, wherein said distance is a set theoretic distance.

17. The computer readable medium of claim 16, wherein said set theoretic distance is calculated by:
determining, for a given gene, a first regulation type associated with exposure to a first condition from said plurality of potential conditions;
determining, for said given gene, a second regulation type associated with exposure to a second condition from said plurality of potential conditions; and
scoring said gene in accordance with a comparison of said first regulation type and said second regulation type.

18. The computer readable medium of claim 17, wherein said scoring comprises:
assigning a lowest score if said first regulation type and said second regulation type are identical;
assigning a highest score if said first regulation type and said second regulation type are disjoint;
assigning a second-lowest score if one of said first regulation type and said second regulation type is a subset of the other; and
assigning a second-highest score if neither of the first regulation type and the second regulation type is a subset of the other.

19. The computer readable medium of claim 15, wherein said distance is a bit-wise distance.

20. The computer readable medium of claim 19, wherein a regulation type for each gene is a vector comprising:
a first bit position corresponding to upregulation;
a second bit position corresponding to downregulation; and
a third bit position corresponding to no change.

21. The computer readable medium of claim 15, wherein said distance is a Hamming distance.

22. The computer readable medium of claim 15 wherein said subset of genes is identified in accordance with a greedy hill-climbing algorithm.

23. The computer readable medium of claim 22, wherein said greedy hill-climbing algorithm comprises:
starting with a group comprising every gene in said set of test data;
removing genes from said group one at a time until said group is empty, based on which gene in said group, at a given time, yields a largest distance vector for a remaining set of genes; and
selecting a number of last-removed genes to comprise said subset.

24. The computer readable medium of claim 22, wherein said greedy hill-climbing algorithm comprises:
starting with an empty group;
adding genes to said group one at a time, based on which gene in said group, at a given time, yields a largest distance vector for said group, which now comprises at least one gene; and
selecting a number of first-added genes to comprise said subset.

25. The computer readable medium of claim 15, wherein each of said pairs of conditions is an ordered pair comprising a first condition and a second condition.

26. The computer readable medium of claim 25, wherein a distance between said first condition and said second condition comprises a sum of:
a number of bits comprising one for a first regulation type associated with said first condition; and
a number of bits comprising zero for a second regulation type associated with said second condition.

27. The computer readable medium of claim 26, wherein a distance between said second condition and said first condition is not necessarily equal to said distance between said first condition and said second condition.

28. The computer readable medium of claim 15, wherein a general ability of said subset of genes to distinguish between any two conditions in said plurality of potential conditions is calculated by:
for each pair of conditions in said plurality of potential conditions, computing a distance therebetween relative to each gene in said subset;
for each pair of conditions, summing said distances over said subset;
identifying a smallest sum of said distances; and
associating said ability with said smallest sum.

29. An apparatus for selecting genes to be analyzed to determine exposure to a condition from among a plurality of potential conditions, comprising:
means for determining, for each gene in a set of test data comprising a plurality of genes and corresponding expression patterns for exposure to said plurality of potential conditions, a distance between each pair of conditions in said plurality of potential conditions; and
means for identifying a subset of genes from within said set of test data for which said distance between each pair of conditions is maximized.
